(19) United States
(12) Patent Application Publication
(10) Pub. No.: US 2025/0283802 A1
(43) Pub. Date: Sep. 11, 2025

(54) SYSTEMS, KITS, AND METHODS FOR DETERMINING CORROSION TENDENCIES OF AN AQUEOUS SAMPLE ON METAL ALLOYS EXPOSED THERETO

(71) Applicant: Field Water Testing, LLC, Bountiful, UT (US)

(72) Inventor: Christopher Lloyd, West Valley City, UT (US)

(21) Appl. No.: 19/070,268

(22) Filed: Mar. 4, 2025

Related U.S. Application Data
(60) Provisional application No. 63/561,687, filed on Mar. 5, 2024.

(51) Int. Cl. G01N 17/00; G01N 33/18

(52) U.S. Cl. CPC G01N 17/006; G01N 33/18

(57) ABSTRACT

Disclosed are a system, kit, and method for assessing the corrosion tendencies of metal alloys in aqueous solutions. In one embodiment, a metal alloy sample, is exposed to a water sample, and the aqueous solution, is tested for indicative metal ions using reagents, that undergo spectral changes. The test compares the metal alloy sample exposed aqueous solution, to an unexposed spectral reference standard, to determine corrosion extent, anticorrosive treatment effectiveness, and optimal treatment concentrations. The kit may include colorogenic and/or fluorogenic reagents, spectral reference standards, and the system may incorporate a dual-detection spectrometry instrument for precise recorded analysis.

### CROSS-REFERENCE TO RELATED APPLICATIONS

This application claims priority to and the benefit of U.S. Provisional Application 63/561,687 titled “Systems, Kits, And Methods For Determining Corrosion Tendencies Of An Aqueous Sample On Metal Alloys Exposed Thereto” and filed on Mar. 5, 2024, which is herein incorporated in its entirety to the extent permissible under applicable patent laws and rules for the relevant jurisdictions.

### FIELD

The subject matter disclosed herein relates to potentially corrosive water samples and particularly relates to systems, kits, and methods for determining corrosion tendencies of an aqueous sample on metal alloys exposed thereto.

### BACKGROUND

Corrosion, which is a process that typically oxidizes metal to metal oxides and/or solubilizes metals into solution, is generally detrimental to equipment, structures and the like. This process can be chemical, microbially induced, galvanic (electrochemical) or heat induced; corrosion can occur through other processes or a mixture of several processes. Prediction of whether an aqueous solution will be corrosive to a selected metal alloy has relied on several chemical models with dubious results.

### BRIEF SUMMARY

In some aspects, the techniques described herein relate to a method for determining corrosion of a metal alloy by an aqueous solution, including: exposing a metal alloy sample to an aqueous solution for a predetermined amount of time; testing the aqueous solution after exposure to the metal alloy sample for a presence and/or a concentration of indicative metal ions released from the metal alloy into the aqueous solution; performing a comparison of the presence and concentration of the indicative metal ions in the aqueous solution after exposure to the metal alloy sample with the presence and/or concentration of the indicative metal ions in the aqueous solution before exposure to the metal alloy sample; determining a corrosive effect of the aqueous solution on the metal alloy based on the comparison.

In various aspects, the techniques described herein relate to a method, wherein the indicative metal ions are a component of the metal alloy.

In certain aspects, the techniques described herein relate to a method, further including using a reaction vial containing one or more reagents formulated to exhibit changes in spectral characteristics based on the presence and/or the concentration of the indicative metal ions.

In one or more aspects, the techniques described herein relate to a method, wherein the changes in spectral characteristics include wavelength and/or relative intensity.

In various aspects, the techniques described herein relate to a method, wherein the one or more reagents include one or more of 2,4,6-tris(2-pyridyl)-s-triazine, 1,10-phenanthroline, and ascorbic acid for iron ions.

In certain aspects, the techniques described herein relate to a method, wherein the one or more reagents include one or more of 2-[5-(2-hydroxy-5-sulfophenyl)-3-phenyl-1-formazyl]benzoic acid for zinc and copper ions.

In one or more aspects, the techniques described herein relate to a method, wherein the one or more reagents include one or more of [2,2′-biquinoline]-4,4′-dicarboxylic acid and sodium ascorbate for copper ions.

In some aspects, the techniques described herein relate to a method, further including: using one or more selected instances of the metal alloy sample treated with predetermined anticorrosive treatments corresponding respectively to the one or more selected instances; and determining based on the corrosive effect of the aqueous solution on the one or more selected instances of the metal alloy sample treated with the predetermined anticorrosive treatments, a relative efficacy of the predetermined anticorrosive treatments.

In various aspects, the techniques described herein relate to a method, further including: using one or more selected instances of the metal alloy sample treated with a selected anticorrosive treatment agent at a range of concentrations corresponding respectively to the one or more selected instances; and determining based on the corrosive effect of the aqueous solution on the one or more selected instances of the metal alloy sample treated with the selected anticorrosive treatment agent at the range of concentrations, the relative efficacy of the range of concentrations of the predetermined anticorrosive treatment.

In certain aspects, the techniques described herein relate to a method, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.

In one or more aspects, the techniques described herein relate to a method, further including: selecting the one or more reagents used to test for the presence of one or more species of microbes based on an expected reaction of the one or more reagents with the or more species of microbes, reaction times of the one or more reagents, and reaction conditions under which a selected aqueous solution is tested, to determine microbial influenced corrosion.

In some aspects, the techniques described herein relate to a system including: a kit for determining corrosion tendencies of an aqueous sample on metal alloys exposed thereto including: one or more reagents formulated to indicate, via changes in a first set of spectral characteristics, a concentration of indicative metal ions in a first aliquot of an aqueous solution exposed to a metal alloy sample for a predetermined period; instructions and/or spectral reference standards for comparing the first set of spectral characteristics of the first aliquot of the aqueous solution exposed to a metal alloy sample for a predetermined period with a second set of spectral characteristics of a second aliquot of the aqueous solution in which exposure to the metal alloy sample is omitted; and one or more spectral reference standards configured to be used for determining a concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

In various aspects, the techniques described herein relate to a system, wherein the system further includes a reaction vial containing the one or more reagents formulated to exhibit changes in spectral characteristics based on presence and/or concentration of the indicative metal ions.

In certain aspects, the techniques described herein relate to a system, wherein the changes in spectral characteristics include wavelength and/or relative intensity.

In one or more aspects, the techniques described herein relate to a system, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.

In some aspects, the techniques described herein relate to a system, wherein the one or more of the reagents are colorigenic or fluorogenic reagents for the indicative metal ions.

In various aspects, the techniques described herein relate to a system, wherein the one or more spectral reference standards include physical instances of a substance exhibiting spectral characteristics corresponding substantially to first set of spectral characteristics of the first aliquot of the aqueous solution exposed to the metal alloy sample.

In certain aspects, the techniques described herein relate to a system, wherein the one or more spectral reference standards include physical instances of a liquid exhibiting spectral characteristics corresponding substantially to first set of spectral characteristics of the first aliquot of the aqueous solution exposed to the metal alloy sample.

In one or more aspects, the techniques described herein relate to a system, further including a spectrometry instrument configured to determine the concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

In some aspects, the techniques described herein relate to a system, wherein the spectrometry instrument is configured to determine a set of spectral characteristics of a reaction between indicative metal ions and the one or more of the reagents that are colorigenic or fluorogenic reagents for selected metal ions.

### BRIEF DESCRIPTION OF THE DRAWINGS

A more particular description of the techniques briefly described above will be rendered by reference to specific embodiments that are illustrated in the appended drawings. Understanding that these drawings depict only some embodiments and are not, therefore, to be considered to be limiting of scope, the embodiments will be described and explained with additional specificity and detail through the use of the accompanying drawings, in which:

FIG. 1 is a schematic block diagram illustrating a system for determining corrosion tendencies of metal alloys exposed to aqueous solutions, according to one or more aspects of the present disclosure;

FIG. 2A depicts results of a working example of system, kit, and method for determining corrosion tendencies of an aqueous solution to a carbon steel metal alloy exposed thereto, according to one or more aspects of the present disclosure;

FIG. 2B depicts spectral characteristics from a working example of system, kit, and method for determining corrosion tendencies of an aqueous solution to a carbon steel metal alloy exposed thereto.

FIG. 3A depicts results of a working example of system, kit, and method for determining corrosion tendencies of an aqueous solution to a brass metal alloy exposed thereto, according to one or more aspects of the present disclosure;

FIG. 3B depicts spectral characteristics from a working example of system, kit, and method for determining corrosion tendencies of an aqueous solution to a brass metal alloy exposed thereto; and

FIG. 4 depicts a flowchart diagram of method for determining corrosion tendencies of an aqueous solution to a metal alloy exposed thereto.

### DETAILED DESCRIPTION

Reference throughout this specification to “one aspect,” “an aspect,” or similar language means that a particular feature, structure, or characteristic described in connection with the aspect is included in at least one implementation. Thus, appearances of the phrases “in one implementation,” “in an implementation,” and similar language throughout this specification may, but do not necessarily, all refer to the same implementation, but mean “one or more but not all implementations” unless expressly specified otherwise. The terms “including,” “comprising,” “having,” and variations thereof mean “including but not limited to,” unless expressly specified otherwise. An enumerated listing of items does not imply that any or all of the items are mutually exclusive unless expressly specified otherwise. The terms “a,” “an,” and “the” also refer to “one or more” unless expressly specified otherwise.

Furthermore, the described features, structures, or characteristics of the aspects or implementations may be combined in any suitable manner. In the following description, numerous specific details are provided, such as examples of programming, software modules, user selections, network transactions, database queries, database structures, hardware modules, hardware circuits, hardware chips, etc., to provide a thorough understanding of aspects and implementations. One skilled in the relevant art will recognize, however, that an implementation may be practiced without one or more of the specific details, or with other methods, components, materials, and so forth. In other instances, well-known structures, materials, or operations are not shown or described in detail to avoid obscuring aspects of the implementation.

Aspects of the disclosed implementations are described below with reference to schematic flowchart diagrams and/or schematic block diagrams of methods, measurement apparatuses, systems, and program products according to examples. It will be understood that some blocks of the schematic flowchart diagrams and/or schematic block diagrams, and combinations of blocks in the schematic flowchart diagrams and/or schematic block diagrams, can be implemented by code. This code may be provided to a processor of a general-purpose computer, special purpose computer, or other programmable data processing measurement apparatus to produce a machine, such that the instructions, which execute via the processor of the computer or other programmable data processing measurement apparatus, create means for implementing the functions/acts specified in the schematic flowchart diagrams and/or schematic block diagrams block or blocks.

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding aspects or implementations. Indeed, some arrows or other connectors may be used to indicate only the logical flow of the depicted example aspect. For instance, an arrow may indicate a waiting or monitoring period of unspecified duration between enumerated steps of the depicted example implementation. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Unless expressly noted or otherwise clear from context, like numbers refer to like elements in all figures, including alternate implementation involving like elements.

As used herein, a list using the conjunction of “and/or” includes any single item in the list or a combination of items in the list. For example, a list of A′s, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single items in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B, and C.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

### Introduction

The present disclosure describes a method and kit for determining if a water solution is corrosive to a metal alloy by comparing tests for indicative metal ions after exposure of the water solution to a metal alloy with tests for said indicative metal ion in the water solution. This comparative test can be used to (1) demonstrate whether a water sample is corrosive to said metal alloy, (2) determine whether an anticorrosive treatment is effective, and (3) determine what concentration of anticorrosive treatments are necessary for an aqueous solution to prevent corrosion.

As used herein, the term “spectral parameters” and “spectral characteristics” refer respectively to any parameters or characteristics which can be observed visually, via spectroscopy, and/or using colorimetry.

Various approaches for determining or indicating if a water solution is corrosive to a metal alloy have been attempted. Such approaches have limitations that reduce the desirability of their use.

One approach is referred to as the Langlier Saturation Index (LSI) which is used as indicator of water scaling and corrosion potential, especially in cooling tower waters. It uses measurements of calcium hardness, alkalinity, pH, total dissolved solids, and temperature to predict the saturation of calcium carbonate (the mineral calcite) and thus is meant to predict limescale formation. Corrosion of carbon steel is predicted by the LSI from solutions that are not either balanced or are not predicted to form scale. The Stiff-Davis Index (SDI) is a modification of the LSI where higher ionic strength is indicated and attempts to compensate for the effect of increased total dissolved solids.

The Ryznar Stability Index (RSI) is also based on calcite equilibrium utilizing measurements of temperature, calcium hardness, alkalinity, total dissolved solids, and pH. This index predicts the corrosion of limescale for aqueous solutions and thus predicts corrosion after the removal of protective scales.

The Puckorius Scaling Index (PSI) measures the buffering capacity of an aqueous solution by calculating the maximum quantity of limescale that is expected to form from a water solution to bring said solution to equilibrium. This index also uses measurements of calcium hardness, alkalinity, pH, total dissolved solids, and temperature to predict scaling tendencies. Corrosion is predicted when scaling is not.

The Larson-Skold Index uses measurements of chloride, sulfate, bicarbonate, and carbonate to predict whether passivated steel is predicted to corrode. (Alternately, measurements of alkalinity and pH can be used to predict carbonate and bicarbonate contributions.) The Chloride Sulfate Mass Ratio (CSMR) is also used in a similar manner.

The Aggressive Index (AI) is determined from measurements of pH, alkalinity, and calcium hardness and is considered more predictive of the corrosivity of water.

More direct measurements of corrosion have been utilized where “corrosion coupons” made of metal alloys are subjected to water samples and the mass loss of said coupons are determined after exposure to the water sample. Loss of some of the original alloy mass indicates corrosion, with greater losses corresponding to greater corrosion. These “corrosion coupons” or “loss coupons” have been employed directly attached to the inside of the pipe, or other apparatuses located separate from main flow lines. Sacrificial probes or stacks are also used to demonstrate mass loss on coupons for the detection and demonstration of corrosion. Recently, wireless corrosion-responsive sensors have been utilized which transmit signals as the sensors themselves are sacrificially corroded.

To provide improvements over existing approaches, the systems, methods, and kits disclosed herein utilize the detection of metal ions from an alloy to demonstrate and detect corrosion by comparing the presence and concentration of said metal ions (indicative metal ions) in the water solution exposed to the alloy to the concentration of indicative metal ions in that water solution which has not been exposed to the alloy.

This way, corrosion is indicated when the alloy-exposed solution exhibits more indicative metal ions as the water solution may have contained said metal ions initially, with the indicative metal ion arising from corrosion of the alloy. The choice of the indicative metal ion utilized depends on the identity of the alloy, with said metal being a component of the alloy. The greater the relative presence of the indicative metal ions to the baseline water sample the larger the corrosion from the water sample. This is demonstrated in the description of the embodiments below.

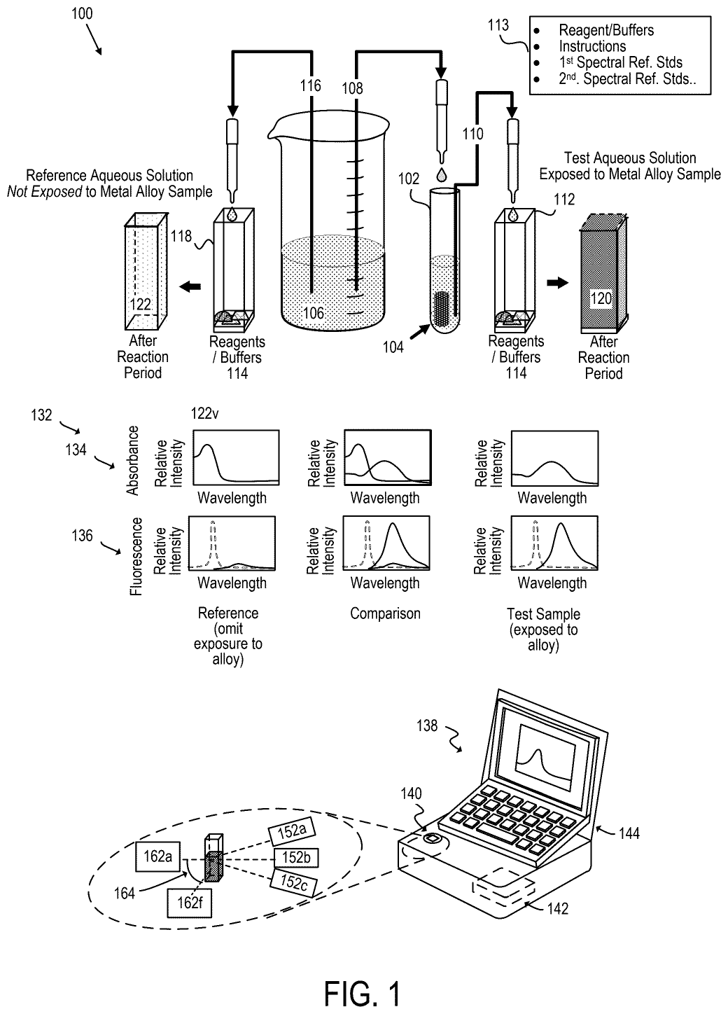

FIG. 1 is a schematic block diagram illustrating a system 100 for determining corrosion tendencies of metal alloys exposed to aqueous solutions, according to one or more aspects of the present disclosure.

The system 100 may include a sample tube 102 containing a passivated metal alloy sample 104 of interest provided for testing of a water sample 106. To the sample tube 102, a known amount of the water sample 106 is added at step 108 for a predetermined amount of time allowing for chemical corrosion to occur (based on times when corrosion occurs if it will occur). Passivated (cleaned) samples are utilized to prevent any indicative metal ions from simply washing off the surface to eliminate false corrosion results. After the specified amount of time, some or all of the water sample that has been in contact with the alloy is added at step 110 to a reaction vial 112 that contains one or more reagents 114 including all the dry ingredients including both ion detection reagents and pH adjustment buffers necessary to test for the first indicative metal ion of the alloy. One or more pH adjustment buffers used to stabilize the pH of the reference aqueous solution and the test aqueous solution, fits within the category of the one or more reagents 114 because they actively participate in maintaining the environmental conditions necessary for the metal alloy corrosive water detection reactions to occur predictably.

At step 116, a similar amount of the water sample 106 is added to an additional reaction vial 118. The reaction vial 118 contains reagents which either change color or exhibit a change in phosphorescence (including fluorescence) when exposed to a second indicative metal ion of the metal alloy. These reaction vials 112, 118 should be allowed to react fully to allow a first indicative metal chemical detection reaction 120 and second indicative metal chemical detection reaction in which exposure to the metal alloy is omitted to enable the results of the reaction to serve as a tangible reference standard 122. A difference in a color change or fluorescence between the water sample which has been exposed to the metal alloy sample 104 depicted in 120 after the reaction period and that of the spectral reference standard 122 after the reaction period indicates the presence of corrosion. In various examples, the spectral reference standard 122 may be a virtual reference standard 122b stored in an instrument such as a spectrometry instrument 138.

In the depicted example system 100, ferrous-based metal alloys (such as cast iron, ferroalloys, steel, and steel alloys) the sample tube 102 contains a sample of the alloy of interest. When the water sample 106 is added, mixing is utilized as ferrous-based metal alloys typically require dissolved oxygen which much be replaced during the desired corrosion assay time due to depletion of the oxidative reactions which occur. The reaction vials 112, 118 contain iron ion reaction reagents and all necessary buffers to detect the presence of iron ions; these reagents include (but are not limited to) 2,4,6-tris(2-pyridyl)-s-triazine, 1,10-phenanthroline, ascorbic acid and the like.

In certain examples, copper, brass, and bronze alloys are used in the sample tube 102 and reaction vials 112, 118 contain one or more reagents and buffers 114 such as 2-[5-(2-hydroxy-5-sulfophenyl)-3-phenyl-1-formazyl]benzoic acid and one or more first pH adjustment buffers selected to facilitate detection of zinc and copper ions and/or [2,2′-biquinoline]-4,4′-dicarboxylic acid and sodium ascorbate one or more second pH adjustment buffers selected to facilitate detection of copper ions.

In some examples, where the one or more reagents 114 include 2-[5-(2-hydroxy-5-sulfophenyl)-3-phenyl-1-formazyl]benzoic acid—the complexation with zinc and copper ions is facilitated by a slightly alkaline environment. A Tris-HCl buffer adjusted to about pH 8.0 may be beneficially used for such chromogenic reagents, as it promotes deprotonation of the phenolic —OH groups and carboxylic acid functionalities to improve metal coordination.

For various example, where [2,2′-biquinoline]-4,4′-dicarboxylic acid is used together with sodium ascorbate for copper ions—a mildly acidic environment may be suitable to facilitate detection. An acetate buffer adjusted to around pH 5.0 may be chosen to keep copper in solution and prevent hydroxide precipitation, while maintaining the stability of both the biquinoline reagent and the ascorbate.

The system 100 includes a kit 113 including: one or more first reagents 114 formulated to indicate, via changes in a first set of spectral characteristics 132, a concentration of indicative metal ions in a first aliquot of an aqueous solution exposed to a metal alloy sample for a predetermined period; instructions and/or spectral reference standards for comparing the first set of spectral characteristics of the first aliquot of the aqueous solution exposed to a metal alloy sample for a predetermined period with a second set of spectral characteristics of a second aliquot of the aqueous solution in which exposure to the metal alloy sample is omitted; and one or more spectral reference standards configured to be used for determining a concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

In one or more examples, the system 100 includes reaction vials 112, 118 containing the one or more reagents and/or buffers 114 formulated to exhibit changes in spectral characteristics 132 based on presence and/or concentration of the indicative metal ions. In some examples, the changes in spectral characteristics 132 may include wavelength and/or relative intensity. The relative intensity may be of absorbance 134 for colorogenic reagents and fluorescence 136 for fluorogenic reagents.

In some examples, the metal alloy sample 104 is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.

In certain examples, one or more reagents are colorigenic reagents or fluorogenic reagents for the indicative metal ions.

In some examples, the system includes one or more spectral reference standards that are physical instances of a substance exhibiting spectral characteristics corresponding substantially to first set of spectral characteristics of the first aliquot of the aqueous solution exposed or not exposed to the metal alloy sample. For example, in FIG. 1, a reference aqueous solution in reaction vial 118 on the left-hand side is not exposed to the metal alloy sample 104 and a test aqueous solution in reaction vial 112 or the right-hand side is exposed to the metal alloy sample 104.

In certain examples, the system 100 further includes spectrometry instrument 138 configured to determine the concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

In various examples, the spectrometry instrument 138 is a dual-detection spectrometry instrument that utilizes a unique configuration of excitation sources 152a, 152b, 152c and two detectors 162a, 162f to facilitate the measurement of absorbance, fluorescence, and scattered light from the aqueous solution exposed to the metal alloy sample. The excitation sources, such as laser diodes, are positioned to direct their beams through the fluid test sample, with their arrangement optimized to provide high-intensity and stable illumination. In some examples the excitation sources 152a, 152b, and 152c are modulated at a fixed frequency to enhance the signal-to-noise ratio, allowing for improved isolation and detection of signals associated with scattered or fluorescent light emissions. In certain examples, a first detector 162a is located on the opposite side of the fluid test sample, in alignment with the illumination angle or path of the excitation sources and is used to measure the absorbance or transmission of light that passes directly through the sample. This configuration enables precise quantification of light attenuation caused by the analytes in the solution.

A second detector 162b is positioned at an offset angle 164 relative to the illumination angle or path of the excitation sources, such that it is generally orthogonal to the direct transmission path or within a suitable range of angles optimized for detecting scattered or fluorescent light. By placing the second detector 162b at the offset angle 164, typically 90 degrees or within a predetermined range of this value, interference from transmitted light is minimized, thereby enhancing the sensitivity to light emitted by fluorescence responses or scattered by the analytes. The angular placement of the second detector 162b can be adjusted depending on the requirements of the specific measurement, allowing flexibility to optimize the detection of fluorescence or scattered light signals.

This arrangement of excitation sources 152a, 152b, 152c and detectors 162a, 162f within the depicted spectrometry instrument 138 enables dual-mode spectrometry, combining absorbance measurements with the capability to detect fluorescence and scattering. The instrument's configuration, which includes angularly offset detection for fluorescence or scattering alongside direct transmission detection, makes it suitable for multi-mode analysis of metal ion reaction analytes in aqueous solutions.

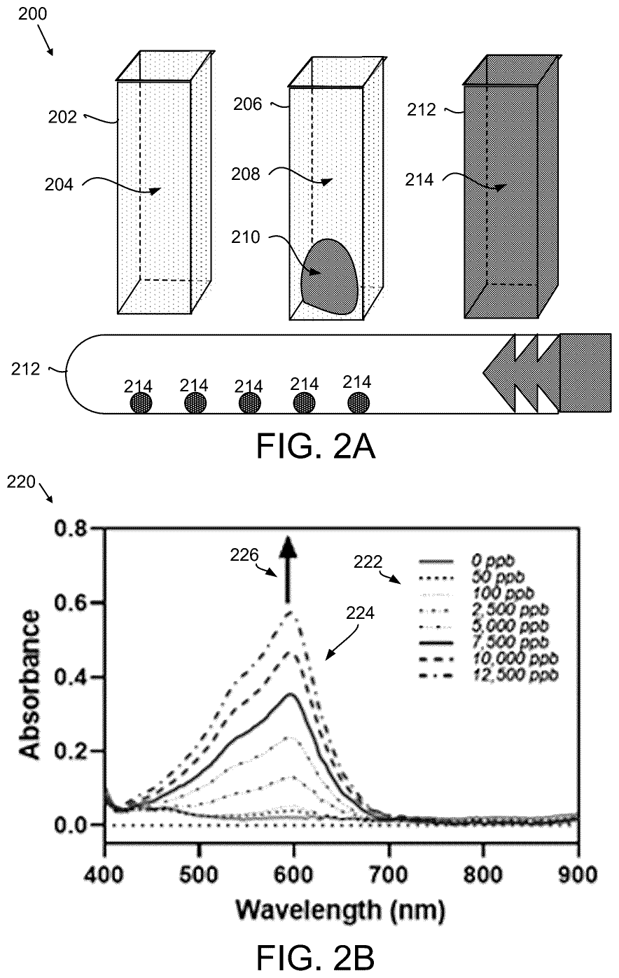

FIG. 2A depicts a first working example of system, kit, and method for determining corrosion tendencies of an aqueous solution to a carbon steel metal alloy exposed thereto, according to one or more aspects of the present disclosure.

In the working example, the indicative metal ions detected are a component of the carbon steel metal alloy. Specifically, the indicative metal ions are iron ions.

In the working example of FIG. 2A, one or more reagents are formulated to exhibit changes in spectral characteristics based on the presence and/or the concentration of the indicative metal ions, i.e., iron ions. In certain examples, such as the working example described with respect to FIG. 2A and FIG. 2B, the one or more reagents include one or more of 2,4,6-tris(2-pyridyl)-s-triazine, 1,10-phenanthroline, ascorbic acid, and a suitable pH adjustment buffer to facilitate detection of iron ions.

In certain examples, the one or more reagent include 2,4,6-tris(2-pyridyl)-s-triazine, 1,10-phenanthroline, ascorbic acid, and a pH adjustment buffer. The pH adjustment buffer may be useful for maintaining an acidic environment—within a pH range of about 3 to 4—to optimize the spectral characteristics of the reaction. For example, an acetate buffer composed of acetic acid and sodium acetate may be used to maintain an acidic condition with a suitable pH range for promoting the effective reduction of Fe(III) to Fe(II) by ascorbic acid and for facilitating the subsequent formation of a colored iron-ligand complex. In certain implementations, a citrate buffer may be employed with a controlled concentration to prevent undue chelation of iron ions. A pH-controlled environment can help reagents remain chemically stable and facilitate spectral changes that are highly attributable to the presence and concentration of iron ions, thereby enhancing the sensitivity and reliability of the detection method.

FIG. 2B depicts the spectral characteristics from a working example of system, kit, and method for determining corrosion tendencies of an aqueous solution to a carbon steel metal alloy exposed thereto. Spectra of these indicative metal vials are shown in FIG. 2B for carbon steel. Iron concentrations of 0, 50, 100, 2500, 5000, 7500, 10,000, and 12,500 parts per billion are with the intensity of a blue color change increasing as the red light is absorbed.

In some examples, one or more fluorogenic reagents suitable for detection corrosion of carbon steel alloy component metal ions such as iron ions may be used in accordance with the disclosed techniques.

For example, in certain implementations, the one or more reagents include fluorogenic reagents for detecting ferrous ions (Fe(II)) released from carbon steel corrosion after a four-hour exposure to an aqueous environment. For example, one suitable fluorogenic reagent may include 9-[2-(bis(2-pyridin-2-ylmethyl)amino)ethyl]-3,6-bis(2-(diethylamino)ethyl)-3H-xanthen-3-one, which exhibits a significant increase in fluorescence upon binding to labile Fe(II) ions. In some example, a fluorogenic reagent such as 3′,6′-bis(diethylamino)-2′,4′,5′,7′-tetrahydroxyspiro[isobenzofuran-1(3H),9′-[9H]xanthen]-3-one may be used, where its fluorescence intensity is modulated in proportion to both the iron and copper ion concentrations. In such implementation, the use of a pH adjustment buffer—such as phosphate buffers prepared from mixtures of sodium phosphate and disodium phosphate—may facilitate maintenance of a suitable pH range to preserve chemical stability of the fluorogenic reagents and enhance binding kinetics with iron ions, thereby providing a sensitive and reliable correlation between fluorescence response and the degree of corrosion on the carbon steel substrate.

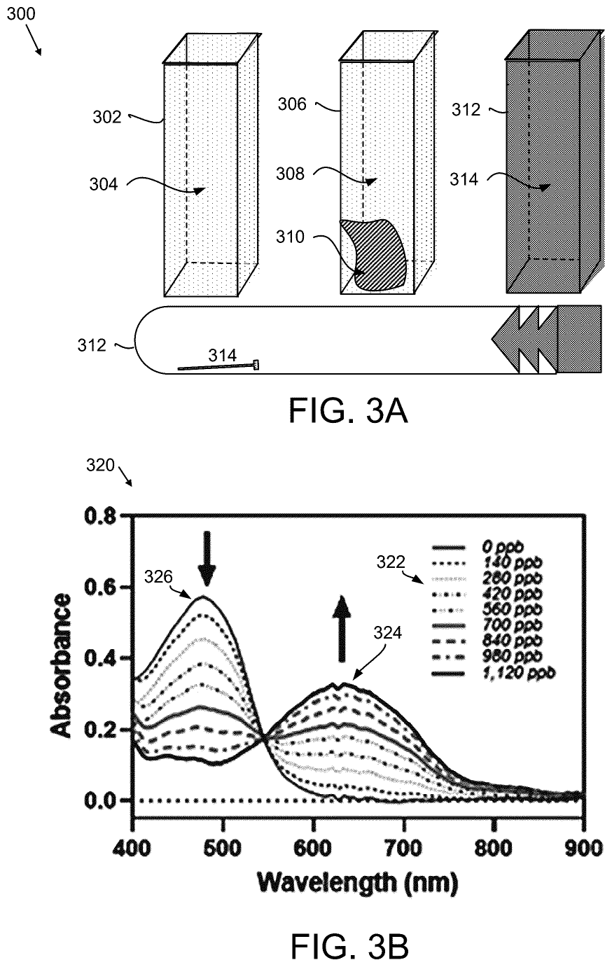

FIG. 3A depicts a second working example of system, kit, and method for determining corrosion tendencies of an aqueous solution to a brass metal alloy (e.g., zinc and copper) exposed thereto.

As mentioned above with respect to FIG. 1., In some examples, for detecting corrosion in a brass metal alloy. In the working example of FIG. 3A, the one or more reagents include 2-[5-(2-hydroxy-5-sulfophenyl)-3-phenyl-1-formazyl]benzoic acid and the complexation with zinc and copper ions is facilitated by a slightly alkaline environment. Accordingly, a pH adjustment buffer is included. For example, a sodium borate buffer adjusted to high pH may be beneficially used for such chromogenic reagents, as it promotes deprotonation of the phenolic —OH groups and carboxylic acid functionalities to improve metal coordination.

Similarly, in various examples, where [2,2′-biquinoline]-4,4′-dicarboxylic acid is used together with sodium ascorbate for copper ions—a basic environment may be suitable to facilitate detection. Sodium borate buffers may be employed.

FIG. 3B depicts spectral characteristics results from the second working example of system, kit, and method for determining corrosion tendencies of an aqueous solution to a brass metal alloy (e.g., zinc and copper).

Results from combined copper and zinc concentrations of 0, 140, 280, 420, 560, 700, 840, 980, and 1,120 parts per billion are shown in FIG. 3B (brass), with the relative intensity of a visually observed orange color in the reaction vial 308 fading as blue light 310 in wavelength ranges from about 450 nm to about 510 nm is absorbed by the reactants 312 and a visually observed blue color increasing as red light 314 is absorbed by the reactants 312.

In certain example, suitable fluorogenic reagents may be used for detecting brass corrosion. Such examples may include one or more fluorogenic reagents such as 2-[2-(bis(carboxymethyl)amino)ethyl]-6-methoxy-3H-xanthen-3-one-9-(2-carboxyphenyl)hydrazide) for detecting zinc ions. Such a reagent exhibits a marked increase in fluorescence upon binding Zn(II) ions, thereby enabling the sensitive detection of zinc released from a brass alloy due to corrosion. Similarly, another reagent such as 2-[2-(bis(pyridin-2-ylmethyl)amino)ethyl]-6-methoxy-3H-xanthen-3-one-9-(2-carboxyphenyl)hydrazide) can provide selectivity and fluorescence enhancement upon Zn(II) binding.

Similarly, for copper ion detection, one or more reagents for detecting brass corrosion may further include one or more reagents such for example, N′-(9-(2-carboxyphenyl)-3,6-dihydroxy-9H-xanthen-9-yl)carbonohydrazide), which is engineered to display minimal fluorescence in its free state but to produce a significant fluorescent signal upon coordination with Cu(II) ions. In various examples, a BODIPY-based probe may be employed, where the core fluorophore is based on 4,4-difluoro-4-bora-3a,4a-diaza-s-indacene, functionalized with suitable copper-binding groups to ensure high sensitivity and selectivity. The combined use of these reagents—optimized as desired with one or more suitable pH adjustment buffers—facilitates a dual-detection assay capable of simultaneously monitoring the release of zinc and copper ions from brass alloys, thereby providing a robust indicator of corrosion.

Additional metal alloy corrosion detection examples according to the disclosed techiniques, may include but are not limited to other metallic alloys including aluminum alloys, cobalt alloys, gold alloys, lead alloys, magnesium alloys, nickel alloys, mischmetals (rare earth alloys), silver alloys, tin alloys, titanium alloys, uranium alloys, zinc alloys, and zirconium alloys with their corresponding indicative metal ion reaction chemistries.

The disclosed corrosion tests can also be performed on waters which have been physically or chemically treated; for instance, the corrosion assays can be performed on water that has been softened by chemical, physical or ion-exchange processes to determine whether softening (removal of alkaline earth metal ions and-or common anions) has resulted in the water becoming corrosive to ferrous-based metals, brass or copper piping, or promotes dezincification of brass connectors and fixtures.

It will also be appreciated by those skilled in the art that the disclosed corrosion testing method can be used on waters that have been treated with anticorrosion chemicals to determine whether said treatments have been effective on waters found to be corrosive to any metal alloy. Additionally, using these corrosion testing methods can be used to optimize the necessary concentrations of anticorrosion chemicals to be used in the treatment of corrosive waters. Using these corrosion detection methods can also be used to demonstrate microbial induced corrosion to corresponding alloys by altering reaction times and conditions.

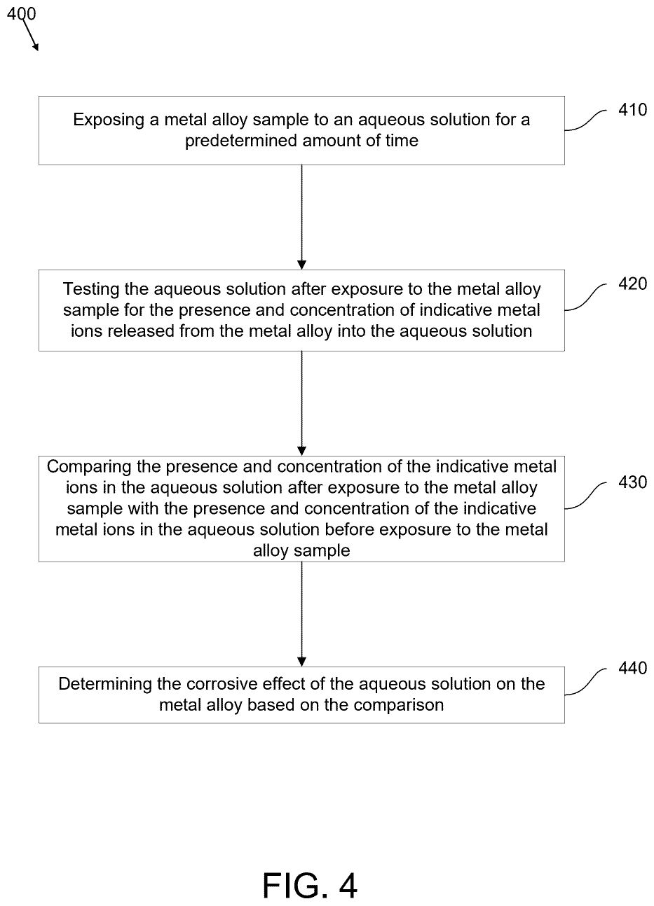

FIG. 4 depicts a flowchart diagram of method 400 for determining corrosion tendencies of an aqueous solution to a metal alloy exposed thereto.

In various examples, the method include exposing 410 a metal alloy sample to an aqueous solution for a predetermined amount of time.

In some examples, the method 400 further includes testing 420 the aqueous solution after exposure to the metal alloy sample for the presence and concentration of indicative metal ions released from the metal alloy into the aqueous solution.

In certain examples, the method 400 include comparing 430 the presence and concentration of the indicative metal ions in the aqueous solution after exposure to the metal alloy sample with the presence and concentration of the indicative metal ions in the aqueous solution before exposure to the metal alloy sample.

In some examples, the method 400 include determining 440 the corrosive effect of the aqueous solution on the metal alloy based on the comparison.

The numbered clauses or statements in the paragraphs below and portions thereof describe various implementations or embodiments of the present disclosure that may be used individually and/or in various combinations with each other or with any subject matter disclosed herein. These implementations are intended to be exemplary and other implementations or combinations may be practiced by a person of ordinary skill using any portion or all of the subject matter disclosed herein for guidance.
Clause 1. A method for determining corrosion of a metal alloy by an aqueous solution, comprising: exposing a metal alloy sample to an aqueous solution for a predetermined amount of time; testing the aqueous solution after exposure to the metal alloy sample for a presence and/or a concentration of indicative metal ions released from the metal alloy into the aqueous solution; performing a comparison of the presence and concentration of the indicative metal ions in the aqueous solution after exposure to the metal alloy sample with the presence and/or concentration of the indicative metal ions in the aqueous solution before exposure to the metal alloy sample; determining a corrosive effect of the aqueous solution on the metal alloy based on the comparison.
Clause 2. The method of clause 1, wherein the indicative metal ions are a component of the metal alloy.
Clause 3. The method of clauses 1 or 2, further comprising using a reaction vial containing one or more reagents formulated to exhibit changes in spectral characteristics based on the presence and/or the concentration of the indicative metal ions.
Clause 4. The method of clause 3, wherein the changes in spectral characteristics comprise wavelength and/or relative intensity.
Clause 5. The method of clauses 3 or 4, wherein the one or more reagents include one or more of 2,4,6-tris(2-pyridyl)-s-triazine, 1,10-phenanthroline, and ascorbic acid for iron ions.
Clause 6. The method of any of clauses 3-5. wherein the one or more reagents include one or more of 2-[5-(2-hydroxy-5-sulfophenyl)-3-phenyl-1-formazyl]benzoic acid for zinc and copper ions.
Clause 7. The method of any of clauses 3-6, wherein the one or more reagents include one or more of [2,2′-biquinoline]-4,4′-dicarboxylic acid and sodium ascorbate for copper ions.
Clause 8. The method of any of clauses 3-7, further comprising: using one or more selected instances of the metal alloy sample treated with predetermined anticorrosive treatments corresponding respectively to the one or more selected instances; and determining based on the corrosive effect of the aqueous solution on the one or more selected instances of the metal alloy sample treated with the predetermined anticorrosive treatments, a relative efficacy of the predetermined anticorrosive treatments.
Clause 9. The method of any of clauses 3-8, further comprising: using one or more selected instances of the metal alloy sample treated with a selected anticorrosive treatment agent at a range of concentrations corresponding respectively to the one or more selected instances; and determining based on the corrosive effect of the aqueous solution on the one or more selected instances of the metal alloy sample treated with the selected anticorrosive treatment agent at the range of concentrations, the relative efficacy of the range of concentrations of the predetermined anticorrosive treatment.
Clause 10. The method of any of clauses 3-9, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.
Clause 11. The method of any of clauses 3-10, further comprising: selecting the one or more reagents used to test for the presence of one or more species of microbes based on an expected reaction of the one or more reagents with the or more species of microbes, reaction times of the one or more reagents, and reaction conditions under which a selected aqueous solution is tested, to determine microbial influenced corrosion.
Clause 12. A system comprising: a kit for determining corrosion tendencies of an aqueous sample on metal alloys exposed thereto comprising: one or more reagents formulated to indicate, via changes in a first set of spectral characteristics, a concentration of indicative metal ions in a first aliquot of an aqueous solution exposed to a metal alloy sample for a predetermined period; instructions and/or spectral reference standards for comparing the first set of spectral characteristics of the first aliquot of the aqueous solution exposed to a metal alloy sample for a predetermined period with a second set of spectral characteristics of a second aliquot of the aqueous solution in which exposure to the metal alloy sample is omitted; and one or more spectral reference standards configured to be used for determining a concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.
Clause 13. The system of clause 12, wherein the system further comprises a reaction vial containing the one or more reagents formulated to exhibit changes in spectral characteristics based on presence and/or concentration of the indicative metal ions.
Clause 14. The system of clause 13, wherein the changes in spectral characteristics comprise wavelength and/or relative intensity.
Clause 15. The system of any of clauses 12-14, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.
Clause 16. The system of any of clauses 12-15, wherein the one or more of the reagents are colorigenic or fluorogenic reagents for the indicative metal ions.
Clause 17. The system of any of clauses 12-16, wherein the one or more spectral reference standards comprise physical instances of a substance exhibiting spectral characteristics corresponding substantially to first set of spectral characteristics of the first aliquot of the aqueous solution exposed to the metal alloy sample.
Clause 18. The system of any of clauses 12-17, wherein the one or more spectral reference standards comprise physical instances of a liquid exhibiting spectral characteristics corresponding substantially to first set of spectral characteristics of the first aliquot of the aqueous solution exposed to the metal alloy sample.
Clause 19. The system of any of clauses 12-18, further comprising a spectrometry instrument configured to determine the concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.
Clause 20. The system of any of clauses 12-19, wherein the spectrometry instrument is configured to determine a set of spectral characteristics of a reaction between indicative metal ions and the one or more of the reagents that are colorigenic or fluorogenic reagents for selected metal ions.
Clause 21. The system of clause 20, wherein the spectrometry instrument is a dual-detection spectrometry instrument comprising: a plurality of modulated excitation sources configured to direct high-intensity beams through the aqueous solution; a first detector aligned with the excitation sources to measure absorbance or transmission; and a second detector positioned at an adjustable offset angle in the range of about 70 degrees to about 110 degrees relative to the excitation sources to detect fluorescence and scattered light.

Example implementations may be practiced in other specific forms. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for determining corrosion of a metal alloy by an aqueous solution, comprising:
exposing a metal alloy sample to an aqueous solution for a predetermined amount of time;
testing the aqueous solution after exposure to the metal alloy sample for a presence and/or a concentration of indicative metal ions released from the metal alloy into the aqueous solution;
performing a comparison of the presence and concentration of the indicative metal ions in the aqueous solution after exposure to the metal alloy sample with the presence and/or concentration of the indicative metal ions in the aqueous solution before exposure to the metal alloy sample; and
determining a corrosive effect of the aqueous solution on the metal alloy based on the comparison.

2. The method of claim 1, wherein the indicative metal ions are a component of the metal alloy.

3. The method of claim 1, further comprising using a reaction vial containing one or more reagents formulated to exhibit changes in spectral characteristics based on the presence and/or the concentration of the indicative metal ions.

4. The method of claim 3, wherein the changes in spectral characteristics comprise wavelength and/or relative intensity.

5. The method of claim 3, wherein the one or more reagents include one or more of 2,4,6-tris(2-pyridyl)-s-triazine, 1,10-phenanthroline, and ascorbic acid for iron ions.

6. The method of claim 3. wherein the one or more reagents include one or more of 2-[5-(2-hydroxy-5-sulfophenyl)-3-phenyl-1-formazyl]benzoic acid and a first pH adjustment buffer for zinc and copper ions.

7. The method of claim 3, wherein the one or more reagents include one or more of [2,2′-biquinoline]-4,4′-dicarboxylic acid, sodium ascorbate, and a second pH adjustment buffer for copper ions.

8. The method of claim 3 further comprising:
using one or more selected instances of the metal alloy sample treated with predetermined anticorrosive treatments corresponding respectively to the one or more selected instances; and
determining based on the corrosive effect of the aqueous solution on the one or more selected instances of the metal alloy sample treated with the predetermined anticorrosive treatments, a relative efficacy of the predetermined anticorrosive treatments.

9. The method of claim 8, further comprising:
using one or more selected instances of the metal alloy sample treated with a selected anticorrosive treatment agent at a range of concentrations corresponding respectively to the one or more selected instances; and
determining based on the corrosive effect of the aqueous solution on the one or more selected instances of the metal alloy sample treated with the selected anticorrosive treatment agent at the range of concentrations, a relative efficacy of the range of concentrations of the predetermined anticorrosive treatment.

10. The method of claim 3, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.

11. The method of claim 3, further comprising:
selecting the one or more reagents used to test for the presence of one or more species of microbes based on an expected reaction of the one or more reagents with the or more species of microbes, reaction times of the one or more reagents, and reaction conditions under which a selected aqueous solution is tested, to determine microbial influenced corrosion.

12. A system comprising:
a kit for determining corrosion tendencies of an aqueous sample on metal alloys exposed thereto comprising:
one or more reagents formulated to indicate, via changes in a first set of spectral characteristics, a concentration of indicative metal ions in a first aliquot of an aqueous solution exposed to a metal alloy sample for a predetermined period;
instructions and/or spectral reference standards for comparing the first set of spectral characteristics of the first aliquot of the aqueous solution exposed to a metal alloy sample for a predetermined period with a second set of spectral characteristics of a second aliquot of the aqueous solution in which exposure to the metal alloy sample is omitted; and
one or more spectral reference standards configured to be used for determining a concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

13. The system of claim 12, wherein the system further comprises a reaction vial containing the one or more reagents formulated to exhibit changes in spectral characteristics based on presence and/or concentration of the indicative metal ions.

14. The system of claim 13, wherein the changes in spectral characteristics comprise wavelength and/or relative intensity.

15. The system of claim 12, wherein the metal alloy is selected from the group consisting of ferrous-based metal alloys, copper, brass, bronze, aluminum alloys, cobalt alloys, gold alloys, lead alloys, magnesium alloys, nickel alloys, mischmetals, silver alloys, tin alloys, titanium alloys, uranium alloys, zinc alloys, and zirconium alloys.

16. The system of claim 12, wherein the one or more of the reagents are colorigenic or fluorogenic reagents for the indicative metal ions.

17. The system of any claim 12, wherein the one or more spectral reference standards comprise physical instances of a substance exhibiting spectral characteristics corresponding substantially to first set of spectral characteristics of the first aliquot of the aqueous solution exposed to the metal alloy sample.

18. The system of any claim 12, wherein the one or more spectral reference standards comprise physical instances of a liquid exhibiting spectral characteristics corresponding substantially to first set of spectral characteristics of the first aliquot of the aqueous solution exposed to the metal alloy sample.

19. The system of any claim 12, further comprising a spectrometry instrument configured to determine the concentration of indicative metal ions based on comparison of the first set of spectral characteristics of the first aliquot of the aqueous solution exposed to a metal alloy sample for a predetermined period with the second aliquot of the aqueous solution in which exposure to the metal alloy sample is omitted.

20. The system of claim 19, wherein the spectrometry instrument is configured to determine a set of spectral characteristics of a reaction between indicative metal ions and the one or more of the reagents that are colorigenic or fluorogenic reagents for selected metal ions.
